(19) United States
(12) Patent Application Publication
(10) Pub. No.: US 2008/0214518 A1
(43) Pub. Date: Sep. 4, 2008

(54) Pharmaceutical Composition Comprising an Anti-Bacterial Agent and an Active Ingredient Selected From Carveol, Thymol, Eugenol, Borneol and Carvacrol

(75) Inventor: Adnane Remmal, Fes (MA)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: ADVANCED SCIENTIFIC DEVELOPMENTS, Casablanca (MA)

(21) Appl. No.: 11/914,015

(22) PCT Filed: May 15, 2006
(86) PCT No.: PCT/IB2006/001350

§ 371 Date: Jan. 22, 2008

(30) Foreign Application Priority Data
May 13, 2005 (IB) PCT/IB2005/001313

(51) Int. Cl. A61K 31/43; A61K 31/545; A61P 31/04

(52) U.S. Cl. 514/192; 514/200

(57) ABSTRACT

The invention relates to a pharmaceutical composition comprising at least one first active therapeutic substance selected among carveol, thymol, eugenol, borneol, carvacrol, alpha-ionone, beta-ionone, as well as isomers, derivatives and mixtures thereof, and comprising at least one second active therapeutic substance that is an antibiotic.

The invention relates to a pharmaceutical composition comprising two therapeutically active substances, one of which exerts a potentiating action on the other, and to the use of said composition.

It is known that the efficacy of therapeutic agents depends on the doses used which, in the case of partial resistance, necessitates increasing the doses of the therapeutic agents in order to attain the desired efficacy. This dose increase leads to problems with adverse effects and acute or chronic toxicity, which may considerably complicate the condition of the treated patients.

Said partial resistance may turn into complete resistance. In this case, increasing the dose no longer has any beneficial therapeutic effect; only the toxic effects are observed. The treatment in such a case consists in changing the therapeutic agent.

This chain of events can repeat itself and lead to the most serious situation: complete resistance to multiple therapeutic agents (multidrug resistance).

For instance, in particular, immunosuppressed patients become increasingly difficult to treat and their life expectancy is correspondingly shortened. Moreover, their quality of life is substantially affected by the administration of high doses of therapeutic agents.

The invention is directed at alleviating these problems by proposing to combine at least two therapeutically active substances, one of which potentiates the activity of the other, which not only makes it possible to lower the doses of each therapeutically active substance but also to treat patients afflicted with infections caused by resistant microorganisms.

In this regard, the invention provides a pharmaceutical composition characterized in that it comprises:
at least one first therapeutically active substance selected from the group consisting of carveol, thymol, eugenol, borneol, carvacrol, alpha-ionone, beta-ionone, and the isomers, derivatives and mixtures thereof,

and,
at least one second therapeutically active substance which is an antibiotic.

The first therapeutic substance can be obtained by chemical synthesis or from a plant source.

Preferably, the antibiotic in the composition of the invention is selected from the group consisting of the beta-lactams, the cephalosporins, fosfomycin, the glycopeptides, the polymyxins, the gramicidins, tyrocidin, the aminosides, the macrolides, the lincosamides, the synergistins, the phenicols, the tetracyclines, fusidic acid, the oxazolidinones, the rifamycins, the quinolones, the fluoroquinolones, the nitrated products, the sulfamides, trimethoprim, and the mixtures thereof.

More preferably, the antibacterial agent is selected from the group consisting of the penicillins, oxacillin, cloxacillin, ampicillin, amoxicillin, bacampicillin, metampicillin, pivampicillin, azlocillin, mezlocillin, piperacillin, ticarcillin, pivmecillinam, sulbactam, tazobactam, imipenem, cephalexin, cephydroxii, cephaclor, cephatrizine, cephalotin, cephapirin, cephazolin, cephoxitin, cephamandole, cephotetan, cephuroxime, cephotaxime, cephsulodin, cephoperazone, cephotiam, cephtazidime, cephtriaxone, cephixime, cephpodoxime, cephepime, latamoxef, aztreonam, vancomycin, vancocin, teicoplanin, polymyxin B, colistin, bacitracin, tyrothricin, streptomycin, kanamycin, tobramycin, amikacin, sisomycin, dibekacin, netilmycin, spectinomycin, spiramycin, erythromycin, josamycin, roxithromycin, clarithromycin, azithromycin, lincomycin, clindamycin, virginiamycin, pristinamycin, dalfopristine-quinupristine, chloramphenicol, thiamphenicol, tetracycline, doxycycline, minocycline, fusidic acid, linezolide, rifamycin, rifampicin, nalidixic acid, oxolinic acid, pipemidic acid, flumequin, pefloxacin, norfloxacin, ofloxacin, ciprofloxacin, enoxacin, sparfloxacin, levofloxacin, moxifloxacin, nitroxolin, tilboquinol, nitrofurantoin, nifuroxazide, metronidazole, ornidazole, sulfadiazine, sulfamethisol, trimethoprim, isoniazide and the derivatives and mixtures thereof. Said antibiotics, and more particularly amoxicillin, can optionally be used in association with clavulanic acid.

A more particularly preferred antibiotic composition is a composition in which the first therapeutically active substance is carveol or carvacrol and the antibiotic is amoxicillin or rifampicin.

Another more particularly preferred antibiotic composition is an antibiotic composition in which the first therapeutically active substance is carveol and the antibiotic is ampicillin, chloramphenicol, tetracycline, streptomycin, erythromycin or polymyxin B.

Yet another more particularly preferred antibiotic composition is an antibiotic composition in which said first therapeutically active substance is alpha-ionone or beta-ionone and the antibiotic is cephazolin.

Still another more particularly preferred antibiotic composition is an antibiotic composition in which said first therapeutically active substance is thymol and the antibiotic is isoniazide.

A Iso, an antibiotic composition in which said first therapeutically active substance is carvacrol and the antibiotic is amoxicillin in association with clavulanic acid is more particularly preferred.

The invention also proposes a kit characterized in that it comprises at least one first container containing a first therapeutically active substance selected from the group consisting of carveol, thymol, eugenol, borneol, carvacrol, alpha-ionone, beta-ionone, and the isomers and derivatives and mixtures thereof, and at least one second container containing a second therapeutically active substance which is an antibiotic.

Lastly the invention proposes a method for treating an infection due to bacteria characterized in that one administers simultaneously or sequentially to a patient having a bacterial infection, at least one first therapeutically active substance selected from the group consisting of carveol, thymol, eugenol, borneol, carvacrol, alpha-ionone, beta-ionone, and the isomers and derivatives and mixtures thereof, and at least one second therapeutically active substance which is an antibiotic.

Preferably, in said method, one simultaneously or sequentially administers to a patient having a bacterial infection between 10 and 200 mg/kg of body weight/day of said first therapeutically active substance, and between 1 and 100 mg/kg of body weight/day of second therapeutically active substance which is an antibiotic.

Preferably, in this method, said first therapeutically active substance is selected from the group consisting of carvacrol, carveol, eugenol, alpha-ionone, beta-ionone and thymol and said second therapeutically active substance is selected from the group consisting of amoxicillin, ampicillin, streptomycin, erythromycin, polymyxin B, chloramphenicol, tetracycline, rifampicin, isoniazide and cephazolin.

The invention will be better understood and other aims and advantages thereof will appear more clearly in the explanatory description which follows and which refers to the single appended FIGURE presenting the results of in vivo tests in mice experimentally infected with a highly resistant strain of Klebsiella pneumoniae and either untreated, or treated with amoxicillin alone, treated with carveol alone, or treated with an antibacterial pharmaceutical composition according to the invention comprising amoxicillin and carveol.

The pharmaceutical composition according to the invention comprises as first therapeutically active substance thymol, eugenol, carvacrol, borneol, carveol, alpha-ionone, beta-ionone, the derivatives and isomers as well as mixtures thereof, in pure form.

Said compounds have well-known antibiotic properties.

Thymol, eugenol, carvacrol, borneol and carveol, alpha-ionone, beta-ionone are found in various proportions in different aromatic plant extracts, that is to say, they can be purified from such plants. However, they can quite simply be obtained by chemical synthesis.

As a matter of fact, the inventors have now discovered that said compounds have a potentiating effect on many therapeutically active substances including known antibiotics which are already used as medicaments specific in this field.

The second therapeutically active substance comprised in the pharmaceutical composition of the invention is therefore an antibiotic, which is already known as such and already used as medicament specific in this field and whose activity is potentiated.

Examples of known antibiotics already used as medicaments specific in this field which can be used in the pharmaceutical composition of the invention, and whose effect will be potentiated by the pure first therapeutically active substance, belong to three families: the beta-lactam family represented by amoxicillin and ampicillin, the cephalosporin family represented by cephazolin, the tetracycline family represented by chlortetracycline, the rifamycin family represented by rifampicin, the peptide family represented by polymyxin, the aminoside family represented by streptomycin, the phenicol family represented by chloramphenicol, the macrolide family represented by erythromycin.

Said compounds can be used alone, or in combination with each other. The derivatives thereof, if they have antibiotic activity, can also be used.

Quite particularly preferred are amoxicillin, optionally in association with clavulanic acid, ampicillin, tetracycline, erythromycin, streptomycin, chloramphenicol, rifampicin, isoniazide, cephazolin and polymyxin B used in combination more particularly with carvacrol, carveol, thymol, alpha-ionone and beta-ionone.

Of course, the pharmaceutical composition according to the invention is not restricted to the use of only those antibiotics mentioned above. In fact, considering the potentiating effect exerted by the first therapeutically active substance defined in the invention, other known or future antibiotics can also be successfully used.

The pharmaceutical composition according to the invention can be formulated so as to be suitable for a simultaneous or sequential administration of said at least first and second therapeutically active substances.

The pharmaceutical form of the pharmaceutical composition of the invention shall be adapted to its use. For example, it can be used in the form of a solution, suspension, tablet or others. The compositions for parenteral administration are generally pharmaceutically acceptable sterile solutions or suspensions which can optionally be prepared immediately before use.

For the preparation of nonaqueous solutions or suspensions, it is possible to use natural vegetable oils like olive oil, sesame oil or paraffin oil or the injectable organic esters such as ethyl oleate. The sterile aqueous solutions can be composed of a solution of therapeutically active substances in water. The aqueous solutions are suitable for intravenous administration in so far as the pH is properly adjusted and they are made isotonic, for example by adding a sufficient amount of sodium chloride or glucose.

In fact, considering the chemical structure of antibiotics, and secondly, considering the chemical structure of carveol, carvacrol, thymol, eugenol, alpha-ionone, beta-ionone, and borneol, it is thought, without intending to be bound by this theory, that carveol, carvacrol, thymol, eugenol, borneol, alpha-ionone and beta-ionone and the isomers, derivatives and mixtures thereof, interact with antibiotics to form complexes having a structure which diffuses more easily into the body's physiological fluids and which diffuses more easily into the cytoplasm of target infected cells.

However, it has been shown that when the different components of the pharmaceutical composition of the invention are mixed in the presence of detergents such as Tween or Triton or solvents such as ethanol or DMSO (dimethyl sulfoxide), the active molecules of the first and second therapeutically active substance associate with the molecules of the detergents and solvents and do not form potentiating complexes.

Now it has been discovered that the potentiating complex forms when an aqueous agar suspension is used, as means of dispersion by viscosity.

Thus, the pharmaceutical composition of the invention will preferably be prepared without detergent and without solvent. For example, it will be prepared as an aqueous suspension made viscous by the addition of agar at a non-solidifying concentration, for example from 1 to 5 grams of agar per liter of suspension.

The pharmaceutical composition of the invention enables the treatment of local or systemic infections caused by resistant microorganisms using doses of each of said first and second therapeutically active substance which are lower than the doses required for treating the same infections due to susceptible microorganisms with one or the other of these same said first and second therapeutically active substances alone. In fact, the composition of the invention enables the use of doses of said first therapeutically active substance, when it is combined with said second therapeutically active substance, which are approximately three to ten times lower than the doses required when said first therapeutically active substance is used alone, and of doses of said second therapeutically active substance, when it is combined with said first therapeutically active substance, which are approximately two to ten times lower than the doses required when said second therapeutically active substance is used alone.

The result is to offer a treatment which has the following advantages:
effective at very low doses against susceptible microorganisms,
effective against microorganisms resistant to a therapeutic agent,
effective against microorganisms resistant to several therapeutic agents,
control of recurrence phenomena,
control of phenomena of resistant microorganisms selection.

In all these cases, there is a notable reduction in the risks of toxicity and/or adverse effects well known to the person of the art, thanks to the potentiation which enables the administration of very low doses.

In addition, the costs of producing the treatment are reduced due to the small quantities of active substances used.

The pharmaceutical compositions according to the invention can be in the form of liposomes or associated with supports such as cyclodextrins or polyethylene glycols.

The pharmaceutical compositions of the invention are a simple and efficient means to combat the problems related to microbial agents in general which comprise mainly resistance to therapeutic agents and toxicity of the latter resulting from the use of high doses.

In fact, carveol, thymol, eugenol, borneol, carvacrol, alpha-ionone and beta-ionone and the derivatives, mixtures and isomers thereof, are simple molecules which have never been described as having any toxicity whatsoever and their addition with its potentiating effect on the second therapeutically active substance enables the use of much lower doses of said second therapeutically active substance.

In a first variant, then, the method for treating patients having a bacterial infection consists in administering to said patients the dose, determined by the physician, of the pharmaceutical composition of the invention comprising suitable doses of at least one said first therapeutically active substance, combined with suitable doses of at least one said second therapeutically substance, that is, the suitable antibiotic.

In a second variant, the method for treating patients having a bacterial infection consists in sequentially administering to said patients the dose determined by the physician of at least one said first therapeutically active substance, followed by the suitable dose of at least one said second therapeutically active substance, that is, the suitable antibiotic, or vice versa.

In this regard, the invention proposes a kit comprising at least one first container containing one of said first therapeutically active substances, and at least one second container containing one of said second therapeutically active substances.

Said kit enables health care personnel to prepare on demand either a mixture of suitable doses of the desired first therapeutic substance(s) and of the desired antibiotic(s), for a simultaneous administration, or to sequentially and separately administer the suitable dose of at least one said first therapeutically active substance, followed by the suitable dose of at least one said second therapeutically active substance, that is, the suitable antibiotic, or vice versa. However, a mixture for simultaneous use shall be preferred in order to allow the potentiation complex to form and to act immediately after administration to the patient.

The invention shall become clearer in the following examples describing different embodiments, which are given for purposes of illustration and not by way of limitation.

### EXAMPLE 1

### Treatment of Different Bacterial Strains with Amoxicillin Potentiated by Carveol (Amox-P)

### In Vitro Test: Determination of Minimal Bactericidal Concentration (MBC) on Different Bacterial Strains

The experiment was carried out with several Gram-negative and Gram-positive bacterial strains with different susceptibilities isolated in the hospital environment. The antibiotic used was amoxicillin, one of the most effective and most widely used antibiotics.

Antibacterial pharmaceutical compositions according to the invention were prepared by mixing amoxicillin at different concentrations with carveol at a sub-inhibitory concentration of 0.3 g per liter of solution or excipient (equivalent to 0.3 mg/ml). The minimal concentration of amoxicillin in combination with carveol 0.3 mg/ml that produced a bactericidal effect was determined. In each case, antibiotic activity was determined either with amoxicillin alone, or with carveol alone, or with the composition of the invention.

Table 1 gives the results of static tests to determine the minimal bactericidal concentration (MBC).

TABLE 1

| | Composition | Composition | |
|---|---|---|---|
| | of the | of the | |
| Bacteria in exponential | invention | invention | Carveol alone |
| growth phase | MBC (μg/ml) | MBC (μg/ml) | MBC (mg/ml) |
| Escherichia coli | >50 | 1 | 2 |
| Salmonella | >50 | 1 | 2 |
| typhimurium | | | |
| Klebsiella pneumoniae | >50 | 5 | 2 |
| Bacillus subtilis | >50 | 1 | 2 |
| Staphylococcus | >50 | 5 | 2 |
| epidermidis | | | |
| Staphylococcus aureus | >50 | 1 | 2 |

Table 1 clearly shows that the composition of the invention had notable bactericidal activity on these strains with different susceptibilities, as compared with amoxicillin alone or with carveol alone.

In fact, it can be seen that by using a carveol concentration of 0.3 mg/ml, which is six times lower than the MBC of carveol alone, the amoxicillin concentration which produced bactericidal activity was at least ten times lower than the concentration of amoxicillin alone capable of exhibiting a bactericidal effect.

### EXAMPLE 2

### In Vivo Tests

Groups of 10 mice were experimentally infected by intraperitoneal injection of 1,000,000 cells (colony-forming units) of amoxicillin-resistant Klebsiella pneumoniae.

The first group was composed of control mice which were infected and untreated.

The second group was composed of infected mice treated by gavage, 24 h post-infection, with amoxicillin alone at a dose of 10 mg/kg of body weight/day.

The third group was composed of infected mice treated by gavage, 24 h post-infection, with carveol alone at a dose of 120 mg/kg of body weight/day.

The fourth group was composed of infected mice treated by gavage, 24 h post-infection, with the pharmaceutical composition of the invention (AMOX-P) at a dose of 2 mg/kg of body weight/day of amoxicillin and 120 mg/kg of body weight/day of carveol.

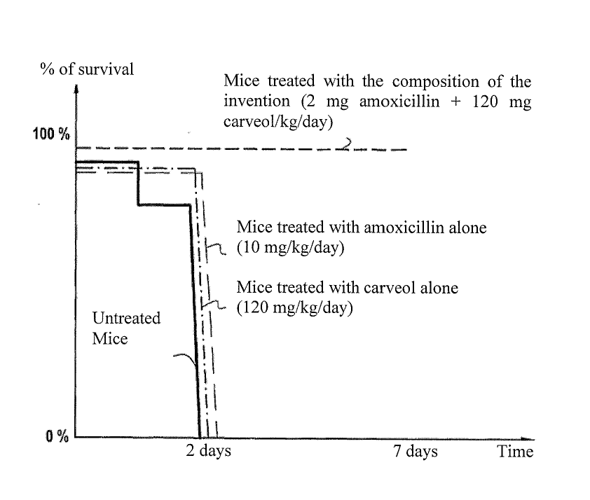

The survival rate was measured over time. The results are given in FIG. 1, which shows that only the mice treated with the pharmaceutical composition were still alive ten days after infection. All the other mice died between the second and third day post-infection.

Examination of the organs of mice which died during the experiment (untreated mice and those treated with amoxicillin alone revealed high loads of Klebsiella pneumoniae in kidney, lung and bone marrow. In contrast, none of the mice treated with the pharmaceutical composition of the invention and sacrificed seven days after stopping treatment showed any bacteria in lung or bone marrow.

In kidney, only three of ten animals still had a very low Klebsiella pneumoniae load corresponding to 10% of that seen in the untreated infected controls.

Consequently, it clearly appears that potentiation of amoxicillin by combining it with carveol as in this example gives surprising results regarding the reduction in the minimal bactericidal concentration in vitro and this potentiation was found in vivo in a model of systemic infection.

Since systemic infections can be life-threatening and are the most difficult forms of infection to treat, especially since they can relapse with selection of increasingly resistant microbes, the pharmaceutical composition of the invention has clearcut advantages.

The method for treating a bacterial infection consists in administering simultaneously or sequentially to a patient having a bacterial infection, the dose determined by the physician of at least one first pure therapeutically active substance selected from the group consisting of carveol, thymol, eugenol, borneol, carvacrol, alpha-ionone, beta-ionone and the isomers, derivatives and mixtures thereof, and the determined dose of at least one second therapeutically substance which is a well-known antibiotic already used in the clinic as medicament specific in this field.

Generally, one simultaneously or sequentially administers to a patient having a bacterial infection between 10 and 200 mg/kg of body weight/day of said first therapeutically active substance, and between 1 and 100 mg/kg of body weight/day of said second therapeutically active substance which is a known antibiotic already used as medicament specific in this field.

### EXAMPLE 3

### Treatment of Different Bacterial Strains with Ampicillin Potentiated by Carveol (Ampi-P)

The experiment was carried out with several resistant bacterial strains isolated in the hospital environment. The antibiotic used was ampicillin, from the beta-lactam family, which is one of the most widely used antibiotics. An antibiotic pharmaceutical composition according to the invention was prepared by mixing ampicillin at different concentrations with carveol at a sub-inhibitory concentration of 0.3 g per liter of solution or excipient. This pharmaceutical composition of the invention was named Ampi-P, for potentiated ampicillin. In each case, antibiotic activity was determined either with ampicillin alone, or with carveol alone, or with the composition of the invention.

Table 2 gives the results of static tests to determine the minimal inhibitory concentration (MIC) and the minimal bactericidal concentration (MBC) in μg/ml.

TABLE 2

| | Ampicillin | | |
|---|---|---|---|
| Bacteria in exponential | alone | Ampi-P | Carveol alone |
| growth phase | MIC (μg/ml) | MBC (μg/ml) | MBC (μg/ml) |
| Escherichia coli | >50 | 5 | 2000 |
| Salmonella typhimurium | >50 | 1 | 2000 |
| Bacillus subtilis | >50 | 10 | 2000 |
| Staphylococcus | >50 | 5 | 2000 |
| epidermidis | | | |

Table 2 shows that the composition of the invention had notable bactericidal activity on the tested strains, as compared with ampicillin alone or with carveol alone.

In fact, it can be seen in Table 2 that by using a carveol concentration of 0.3 mg/ml, which is 6.6 times lower than the MBC of carveol alone, the amoxicillin concentration which produced bactericidal activity was 5 to 50 times lower than the concentration of amoxicillin alone capable of exhibiting a bacteriostatic effect.

Thus, the potentiation of ampicillin by carveol not only allowed a large reduction in the ampicillin dose but also provided bactericidal action at a low dose.

### EXAMPLE 4

### Treatment of Different Bacterial Strains with a Cephalosporin Potentiated by Alpha-Ionone and Beta-Ionone (Cepha-P)

The experiment was carried out with several resistant bacterial strains isolated in the hospital environment. The antibiotic used was cephazolin, from the cephalosporin family, another class of beta-lactams which are among the most widely used antibiotics. An antibiotic pharmaceutical composition according to the invention was prepared by mixing cephazolin at different concentrations with alpha-ionone and beta-ionone at a sub-inhibitory concentration of 0.3 g per liter of solution or excipient. This pharmaceutical composition of the invention was named Cepha-P, for potentiated cephazolin. In each case, antibiotic activity was determined either with cephazolin alone, or with alpha-ionone or beta-ionone alone, or with the composition of the invention.

Table 3 gives the results of static tests to determine the minimal inhibitory concentration (MIC) and the minimal bactericidal concentration (MBC) in μg/ml.

TABLE 3

| | | | | Alpha- | Beta- |
|---|---|---|---|---|---|
| | | | | ionone | ionone |
| Bacteria in | | Cepha-P | Cepha-P | alone | alone |
| exponential | Cephazolin alone | Alpha-ionone | Beta-ionone | MBC | MBC |
| growth phase | MIC (μg/ml) | MBC (μg/ml) | MBC (μg/ml) | (μg/ml) | (μg/ml) |
| Escherichia coli | >50 | 5 | 5 | 2000 | 2000 |
| Salmonella | >50 | 5 | 5 | 2000 | 2000 |
| typhimurium | | | | | |
| Bacillus subtilis | >50 | 5 | 5 | 2000 | 2000 |
| Staphylococcus | >50 | 5 | 5 | 2000 | 2000 |
| epidermidis | | | | | |

Table 3 shows that the composition of the inventions had notable bactericidal activity on the tested strains, as compared with cephazolin alone or with alpha-ionone or beta-ionone alone.

In fact, it can be seen in Table 3 that by using alpha-ionone or beta-ionone at a concentration of 0.3 mg/ml, which is 6.6 times lower than the MBC of alpha-ionone or beta-ionone alone, the cephazolin concentration which produced bactericidal activity was at least ten times lower than the concentration of cephazolin alone capable of exhibiting a bacteriostatic effect.

Thus, the potentiation of cephazolin by alpha-ionone or beta-ionone not only allowed a large reduction in the cephazolin dose but also provided bactericidal action at a low dose.

### EXAMPLE 5

### Treatment of Different Bacterial Strains with Polymyxin B Potentiated by Carveol (Polymix-P)

The experiment was carried out with several resistant bacterial strains isolated in the hospital environment. The antibiotic used was polymyxin B, from the peptide family, which is one of the oldest antibiotics. An antibiotic pharmaceutical composition according to the invention was prepared by mixing polymyxin B at different concentrations with carveol at a sub-inhibitory concentration of 0.3 g per liter of solution or excipient. This pharmaceutical composition of the invention was named Polymix-P, for potentiated polymyxin B. In each case, antibiotic activity was determined either with polymyxin B alone, or with carveol alone, or with the composition of the invention.

Table 4 gives the results of static tests to determine the minimal inhibitory concentration (MIC) and the minimal bactericidal concentration (MBC) in μg/ml.

TABLE 4

| | Polymyxin | | |
|---|---|---|---|
| Bacteria in exponential | B alone | Polymix-P | Carveol alone |
| growth phase | MIC (μg/ml) | MBC (μg/ml) | MBC (μg/ml) |
| Escherichia coli | >50 | 10 | 2000 |
| Salmonella typhimurium | 25 | 10 | 2000 |
| Bacillus subtilis | >50 | 5 | 2000 |
| Staphylococcus | >50 | 5 | 2000 |
| epidermidis | | | |

Table 4 snows that the composition of the invention had notable bactericidal activity on the tested strains, as compared with polymyxin B alone or with carveol alone.

In fact, it can be seen in Table 4 that by using a carveol concentration of 0.3 mg/ml, which is 6.6 times lower than the MBC of carveol alone, the polymyxin B concentration which produced bactericidal activity was 2.5 to 10 times lower than the concentration of polymyxin B alone capable of exhibiting a bactericidal effect.

Thus, the potentiation of polymyxin B by carveol not only allowed a large reduction in the polymyxin B dose effective against resistant Gram-negative bacteria (E. coli and S. typhimurium) but also enlarged its activity spectrum to Gram-positive bacteria (S. epidermidis, B. subtilis) not normally susceptible to polymyxin B.

### EXAMPLE 6

### Treatment of Different Bacterial Strains with Chloramphenicol Potentiated by Carveol (Chloram-P)

The experiment was carried out with several resistant bacterial strains isolated in the hospital environment. The antibiotic used was chloramphenicol, from the phenicol family, which is one of the oldest antibiotics. An antibiotic pharmaceutical composition according to the invention was prepared by mixing chloramphenicol at different concentrations with carveol at a sub-inhibitory concentration of 0.3 g per liter of solution or excipient. This pharmaceutical composition of the invention was named Chloram-P, for potentiated chloramphenicol. In each case, antibiotic activity was determined either with chloramphenicol alone, or with carveol alone, or with the composition of the invention.

Table 5 gives the results of static tests to determine the minimal inhibitory concentration (MIC) and the minimal bactericidal concentration (MBC) in μg/ml.

TABLE 5

| Bacteria | Chloramphenicol | | |
|---|---|---|---|
| in exponential | alone | Chloram-P | Carveol alone |
| growth phase | MIC (μg/ml) | MBC (μg/ml) | MBC (μg/ml) |
| Escherichia coli | >50 | 2 | 2000 |
| Salmonella | >50 | 1 | 2000 |
| typhimurium | | | |
| Bacillus subtilis | >50 | 5 | 2000 |
| Staphylococcus | >50 | 5 | 2000 |
| epidermidis | | | |

Table 5 shows that the composition of the invention had notable bactericidal activity on the tested strains, as compared with chloramphenicol alone or with carveol alone.

In fact, it can be seen in Table 5 that by using a carveol concentration of 0.3 mg/ml, which is 6.6 times lower than the MBC of carveol alone, the chloramphenicol concentration which produced bactericidal activity was 10 to 50 times lower than the concentration of chloramphenicol alone capable of exhibiting a bactericidal effect.

Thus, the potentiation of chloramphenicol by carveol not only allowed a large reduction in the chloramphenicol dose effective against resistant Gram-negative bacteria (E. coli and S. typhimurium) but also enlarged its activity spectrum to Gram-positive bacteria (S. epidermidis, B. subtilis) for which its action is normally only bacteristatic.

### EXAMPLE 7

### Treatment of Different Bacterial Strains with Chlortetracycline Potentiated by Carveol (Tetra-P)

The experiment was carried out with several resistant bacterial strains isolated in the hospital environment. The antibiotic used was chlortetracycline, one of the oldest antibiotics. An antibiotic pharmaceutical composition according to the invention was prepared by mixing chlortetracycline at different concentrations with carveol at a sub-inhibitory concentration of 0.3 g per liter of solution or excipient. This pharmaceutical composition of the invention was named Tetra-P, for potentiated chlortetracycline. In each case, antibiotic activity was determined either with chlortetracycline alone, or with carveol alone, or with the composition of the invention.

Table 6 gives the results of static tests to determine the minimal inhibitory concentration (MIC) and the minimal bactericidal concentration (MBC) in μg/ml.

TABLE 6

| Bacteria | Chlortetracycline | | |
|---|---|---|---|
| in exponential | alone | Tetra-P | Carveol alone |
| growth phase | MIC (μg/ml) | MBC (μg/ml) | MBC (μg/ml) |
| Escherichia coli | >50 | 2 | 2000 |
| Salmonella | >50 | 1 | 2000 |
| typhimurium | | | |
| Bacillus subtilis | >50 | 2 | 2000 |
| Staphylococcus | >50 | 2 | 2000 |
| aureus | | | |

Table 6 snows that the composition of the invention had notable bactericidal activity on the tested strains, as compared with chlortetracycline alone or with carveol alone.

In fact, it can be seen in Table 6 that by using a carveol concentration of 0.3 mg/ml, which is 6.6 times lower than the MBC of carveol alone, the chlortetracycline concentration which produced bactericidal activity was 25 to 50 times lower than the concentration of chlortetracycline alone capable of exhibiting a bactericidal effect.

Thus, the potentiation of tetracycline by carveol not only allowed a large reduction in the chlortetracycline dose but also provided bactericidal action at a very low dose.

### EXAMPLE 8

### Treatment of Different Bacterial Strains with Streptomycin Potentiated by Carveol (Strepto-P)

The experiment was carried out with several resistant bacterial strains isolated in the hospital environment. The antibiotic used was streptomycin, an important member of the aminoside family which is among the most important antibiotics. An antibiotic pharmaceutical composition according to the invention was prepared by mixing streptomycin at different concentrations with carveol at a sub-inhibitory concentration of 0.3 g per liter of solution or excipient. This pharmaceutical composition of the invention was named Strepto-P, for potentiated streptomycin. In each case, antibiotic activity was determined either with streptomycin alone, or with carveol alone, or with the composition of the invention.

Table 7 gives the results of static tests to determine the minimal inhibitory concentration (MIC) and the minimal bactericidal concentration (MBC) in μg/ml.

TABLE 7

| | Streptomycin | | |
|---|---|---|---|
| Bacteria in exponential | alone | Strepto-P | Carveol alone |
| growth phase | MIC (μg/ml) | MBC (μg/ml) | MBC (μg/ml) |
| Escherichia coli | >50 | 5 | 2000 |
| Salmonella typhimurium | >50 | 5 | 2000 |
| Bacillus subtilis | >50 | 5 | 2000 |
| Staphylococcus aureus | >50 | 5 | 2000 |

Table 7 shows that the composition of the invention had notable bactericidal activity on the tested strains, as compared with streptomycin alone or with carveol alone.

In fact, it can be seen in Table 7 that by using a carveol concentration of 0.3 mg/ml, which is 6.6 times lower than the MBC of carveol alone, the streptomycin concentration which produced bactericidal activity was at least 10 times lower than the concentration of streptomycin alone capable of exhibiting a bactericidal effect.

Thus, the potentiation of streptomycin by carveol not only allowed a large reduction in the streptomycin dose but also provided bactericidal action at a very low dose.

### EXAMPLE 9

### Treatment of Different Bacterial Strains with Erythromycin Potentiated by Carveol (Erythro-P)

The experiment was carried out with several resistant bacterial strains isolated in the hospital environment. The antibiotic used was erythromycin, an important member of the macrolide family which is among the most important antibiotics. An antibiotic pharmaceutical composition according to the invention was prepared by mixing erythromycin at different concentrations with carveol at a sub-inhibitory concentration of 0.3 g per liter of solution or excipient. This pharmaceutical composition of the invention was named Erythro-P, for potentiated erythromycin. In each case, antibiotic activity was determined either with erythromycin alone, or with carveol alone, or with the composition of the invention.

Table 8 gives the results of static tests to determine the minimal inhibitory concentration (MIC) and the minimal bactericidal concentration (MBC) in μg/ml.

TABLE 8

| | Erythromycin | | |
|---|---|---|---|
| Bacteria in exponential | alone | Erythro-P | Carveol alone |
| growth phase | MIC (μg/ml) | MBC (μg/ml) | MBC (μg/ml) |
| Escherichia coli | >50 | 10 | 2000 |
| Salmonella | >50 | 10 | 2000 |
| typhimurium | | | |
| Bacillus subtilis | >50 | 15 | 2000 |
| Staphylococcus aureus | >50 | 25 | 2000 |

Table 8 shows that the composition of the invention had notable bactericidal activity on the tested strains, as compared with erythromycin alone or with carveol alone.

In fact, it can be seen in Table 8 that by using a carveol concentration of 0.3 mg/ml, which is 6.6 times lower than the MBC of carveol alone, the erythromycin concentration which produced bactericidal activity was at least two to five times lower than the concentration of erythromycin alone capable of exhibiting a bactericidal effect.

Thus, the potentiation of erythromycin by carveol allowed a large reduction in the erythromycin dose capable of exhibiting a bactericidal effect.

### EXAMPLE 10

### Treatment of Different Mycobacterial Strains with Rifampicin Potentiated by Carvacrol (Rifam-P)

The experiment was carried out with two resistant mycobacterial strains isolated in a veterinary environment. The antibiotic used was rifampicin, an important member of the antitubercular family. An antibiotic pharmaceutical composition according to the invention was prepared by mixing rifampicin at different concentrations with carvacrol at a sub-inhibitory concentration of 0.3 g per liter of solution or excipient. This pharmaceutical composition of the invention was named Rifam-P, for potentiated rifampicin. In each case, antibiotic activity was determined either with rifampicin alone, or with carvacrol alone, or with the composition of the invention.

Table 9 gives the results of static tests to determine the minimal inhibitory concentration (MIC) and the minimal bactericidal concentration (MBC) in μg/ml.

TABLE 9

| | Rifampicin | | |
|---|---|---|---|
| Bacteria in exponential | alone | Rifam-P | Carvacrol alone |
| growth phase | MIC (μg/ml) | MBC (μg/ml) | MBC (μg/ml) |
| Mycobacterium fleii | >75 | 2.5 | 1000 |
| Mycobacterium fortuitum | >75 | 5 | 1000 |

Table 9 shows that the composition of the invention had notable bactericidal activity on the mycobacterial strains tested, as compared with rifampicin alone or with carvacrol alone.

In fact, it can be seen in Table 9 that by using a carvacrol concentration of 0.3 mg/ml, which is 3.3 times lower than the MBC of carvacrol alone, the rifampicin concentration which produced bactericidal activity was at least 25 times lower than the concentration of rifampicin alone capable of exhibiting a bactericidal effect.

Thus, the potentiation of rifampicin by carvacrol enabled a considerable reduction in the rifampicin dose displaying bactericidal activity against rapidly growing mycobacteria not normally susceptible to rifampicin.

### EXAMPLE 11

### Treatment of Different Mycobacterial Strains with Isoniazide Potentiated by Thymol (Izon-P)

The experiment was carried out with two resistant mycobacterial strains isolated in a veterinary environment. The antibiotic used was isoniazide, an important member of the antitubercular family. An antibiotic pharmaceutical composition according to the invention was prepared by mixing isoniazide at different concentrations with thymol at a sub-inhibitory concentration of 0.3 g per liter of solution or excipient. This pharmaceutical composition of the invention was named Izon-P, for potentiated isoniazide. In each case, antibiotic activity was determined either with isoniazide alone, or with thymol alone, or with the composition of the invention.

Table 10 gives the results of static tests to determine the minimal inhibitory concentration (MIC) and the minimal bactericidal concentration (MBC) in μg/ml.

TABLE 10

| | Isoniazide | | |
|---|---|---|---|
| Bacteria in exponential | alone | Izon-P | Carvacrol alone |
| growth phase | MIC (μg/ml) | MBC (μg/ml) | MBC (μg/ml) |
| Mycobacterium fleii | >50 | 1 | 1000 |
| Mycobacterium fortuitum | >50 | 2 | 1000 |

Table 10 snows that the composition of the invention had notable bactericidal activity on the mycobacterial strains tested, as compared with isoniazide alone or with thymol alone.

In fact, it can be seen in Table 10 that by using a thymol concentration of 0.3 mg/ml, which is 3.3 times lower than the MBC of thymol alone, the isoniazide concentration which produced bactericidal activity was at least 25 times lower than the concentration of isoniazide alone capable of exhibiting a bactericidal effect.

Thus, the potentiation of isoniazide by thymol enabled a considerable reduction in the isoniazide dose displaying bactericidal activity against rapidly growing mycobacteria not normally susceptible to isoniazide.

### EXAMPLE 12

### Selection of Resistant Mutants in the Presence of Amoxicillin Potentiated by Carvacrol

The following experiment was carried out in order to confirm that the potentiation disclosed in the invention could prevent the selection of resistant mutants.

Escherichia coli strains susceptible to 5 μg/ml amoxicillin were cultured in the presence of a sub-inhibitory concentration of 3 μg/ml, then seeded into nutrient medium (Muller Hinton) containing increasing concentrations of amoxicillin (4 then 5 then 6 μg/ml . . . ). The same procedure was carried out with amoxicillin potentiated by carvacrol at a concentration two times lower than the MIC of carvacrol alone, namely 500 μg/ml. This experiment was based on the principle that at each subcloning, a mutant resistant to the new amoxicillin concentration would multiply, giving rise to a strain more resistant than the strain from which the subcloning arose.

The results of this experiment are reported in Table 11 showing the selection of mutants with increasing resistance and the number of subclonings needed to obtain them.

TABLE 11

| | Initial | | Median | | Final |
|---|---|---|---|---|---|
| | conc. | No. of | conc. | No. of | conc. |
| | (μg/ml) | subclonings | (μg/ml) | subclonings | (μg/ml) |
| Amoxicillin | 3 | 4 | 17 | 9 | 50 |
| alone | | | | | |
| Potentiated | 3 | 14 | 17 | / | / |
| amoxicillin | | | | | |

Table 11 indicates that with the composition of the invention, 14 successive subclonings were required to select a mutant resistant to an Amox-P concentration of 17 μg/ml, starting from 3 μg/ml, whereas with amoxicillin alone, mutants resistant to 17 μg/ml were obtained in only four subclonings. Mutants with even higher resistance ranging up to 50 μg/ml of amoxicillin alone were obtained after nine subclonings, while with Amox-P, no mutants resistant to a concentration greater than 17 μg/ml were selected.

In fact, these data show, on the one hand, that by using a carvacrol concentration of 0.5 mg/ml, which is two times lower than the MIC of carvacrol alone, it was much more difficult to select resistant mutants with the composition of the invention, as compared with amoxicillin alone (14 subclonings versus 4 subclonings, respectively). Furthermore, the selection of mutants resistant to amoxicillin alone continued ever more easily up to 50 μg/ml, the highest concentration in the experiment, whereas in the presence of the composition of the invention, the resistance reached a plateau at 17 μg/ml.

Thus, it can be seen that potentiation of amoxicillin by carvacrol considerably decreased the possibility of selecting resistant mutants.

### EXAMPLE 13

### Selection of Resistant Mutants in the Presence of Amoxicillin+Clavulanic Acid Potentiated by Carvacrol

The combination was prepared at a proportion of 1 gram of amoxicillin for 0.125 g of clavulanic acid.

The following experiment was carried out in order to confirm that the potentiation disclosed in the invention could prevent the selection of resistant mutants.

Escherichia coli strains susceptible to the amoxicillin/clavulanic acid combination at a concentration of 5 μg/ml amoxicillin were cultured in the presence of a sub-inhibitory concentration of 3 μg/ml, then seeded into nutrient medium (Muller Hinton) containing increasing concentrations of the amoxicillin/clavulanic acid combination (4 then 5 then 6 μg/ml . . . ). The same procedure was carried out with amoxicillin/clavulanic acid potentiated by carvacrol at a concentration two times lower than the MIC of carvacrol alone, namely 500 μg/ml. This experiment was based on the principle that at each subcloning, a mutant resistant to the new amoxicillin/clavulanic acid concentration would multiply, giving rise to a strain more resistant than the strain from which the subcloning arose.

The results of this experiment are reported in Table 12 showing the selection of mutants with increasing resistance and the number of subclonings needed to obtain them.

TABLE 12

| | Initial | | Median | | Final |
|---|---|---|---|---|---|
| | conc. | No. of | conc. | No. of | conc. |
| | (μg/ml) | subclonings | (μg/ml) | subclonings | (μg/ml) |
| Amoxicillin/ | 3 | 8 | 20 | 13 | 50 |
| clavulanic | | | | | |
| acid alone | | | | | |
| Potentiated | 3 | 17 | 20 | / | / |
| amoxicillin/ | | | | | |
| clavulanic | | | | | |
| acid | | | | | |

Table 12 indicates that with the composition of the invention, 17 successive subclonings were required to select a mutant resistant to a 20 μg/ml concentration, starting from 3 μg/ml, whereas with amoxicillin/clavulanic acid alone, mutants resistant to 20 μg/ml were obtained in only eight subclonings. Mutants with even higher resistance ranging up to 50 μg/ml of amoxicillin/clavulanic acid alone were obtained after 13 subclonings, while with the composition of the invention, no mutants resistant to a concentration greater than 20 μg/ml were selected.

In fact, these data show, on the one hand, that by using a carvacrol concentration of 0.5 mg/ml, which is two times lower than the MIC of carvacrol alone, it was much more difficult to select resistant mutants with the composition of the invention, as compared with amoxicillin/clavulanic acid alone (17 subclonings versus 8 subclonings, respectively). Furthermore, the selection of mutants resistant to amoxicillin/clavulanic acid alone continued ever more easily up to 50 μg/ml, the highest concentration in the experiment, whereas in the presence of the composition of the invention, the resistance reached a plateau at 20 μg/ml.

Thus, it can be seen that potentiation of amoxicillin/clavulanic acid by carvacrol considerably decreased the possibility of selecting resistant mutants.

It clearly appears from all of the above examples that the potentiation of antibiotics by said first therapeutically active substances makes it possible to reduce the doses required to fight resistant bacteria, to enlarge the spectrum of activity of the antibiotics, to transform the bacteriostatic effect into a bactericidal action and to make it much more difficult for resistant mutants to emerge.

Of course, the invention is in no way restricted to the embodiments described and illustrated herein which are given solely by way of example.

On the contrary, the invention comprises all the technical equivalents of the methods described herein as well as the combinations thereof where such are carried out in the spirit of the invention.

1-13. (canceled)

14. A pharmaceutical composition comprising:
at least one first therapeutically active substance selected from the group consisting of carveol, thymol, eugenol, borneol, carvacrol, alpha-ionone, beta-ionone, and isomers, derivatives or mixtures thereof; and
at least one second therapeutically active substance which is an antibiotic selected from the group consisting of beta-lactams, cephalosporins, tetracyclines, rifamycins, peptides, amino sides, phenicols, macrolides, and derivatives or mixtures thereof.

15. The composition according to claim 14, wherein said second therapeutically active substance is selected from the group consisting of amoxicillin, amoxicillin in association with clavulanic acid, ampicillin, streptomycin, erythromycin, polymyxin B, chloramphenicol, tetracycline, chlortetracycline, rifampicin, isoniazide, cephazolin, and derivatives or mixtures thereof.

16. The composition according to claim 14, wherein said first therapeutically active substance is selected from the group consisting of carvacrol, carveol, thymol, alpha-ionone and beta-ionone and said second therapeutically active substance is selected from the group consisting of amoxicillin, amoxicillin in association with clavulanic acid, ampicillin, streptomycin, erythromycin, polymyxin B, chloramphenicol, tetracycline, chlortetracycline, rifampicin, isoniazide, cephazolin, and derivatives or mixtures thereof.

17. The composition according to claim 16, wherein said first therapeutically active substance is carveol or carvacrol and second therapeutically active substance is selected from the group consisting of amoxicillin, ampicillin, chloramphenicol, tetracycline, chlortetracycline, streptomycin, erythromycin, polymyxin B, rifampicin, amoxicillin in association with clavulanic acid, and derivatives and mixtures thereof.

18. The composition according to claim 17, wherein said first therapeutically active substance is carveol and second therapeutically active substance is amoxicillin.

19. The composition according to claim 16, wherein said first therapeutically active substance is alpha-ionone or beta-ionone and second therapeutically active substance is cephazolin.

20. The composition according to claim 16, wherein said first therapeutically active substance is thymol and said second therapeutically active substance is isoniazide.

21. The composition according to claim 16, wherein said first and second therapeutically active substances are suspended in an aqueous agar solution.

22. The composition according to claim 16, wherein said composition does not include any detergent or solvent.

23. A kit comprising:
at least one first container containing a first therapeutically active substance selected from the group consisting of carveol, thymol, eugenol, borneol, carvacrol, alpha-ionone, beta-ionone and isomers, derivatives or mixtures thereof; and
at least one second container containing a second therapeutically active substance which is an antibiotic selected from the group consisting of beta-lactams, cephalosporins, tetracyclines, rifamycins, peptides, aminosides, phenicols, macrolides and derivatives or mixtures thereof.

24. The kit according to claim 23, wherein said second therapeutically active substance selected from the group consisting of amoxicillin, amoxicillin in association with clavulanic acid, ampicillin, streptomycin, erythromycin, polymyxin B, chloramphenicol, tetracycline, chlortetracycline, rifampicin, isoniazide, cephazolin, and derivatives or mixtures thereof.

25. The kit according to claim 23, wherein said first therapeutically active substance is selected from the group consisting of carvacrol, carveol, thymol, alpha-ionone and beta-ionone and said second therapeutically active substance is selected from the group consisting of amoxicillin, amoxicillin in association with clavulanic acid, ampicillin, streptomycin, erythromycin, polymyxin B, chloramphenicol, tetracycline, chlortetracycline, rifampicin, isoniazide, cephazolin, and derivatives or mixtures thereof.

26. The kit according to claim 25, wherein said first therapeutically active substance is carveol or carvacrol and said second therapeutically active substance is selected from the group consisting of amoxicillin, ampicillin, chloramphenicol, tetracycline, chlortetracycline, streptomycin, erythromycin, polymyxin B, rifampicin, amoxicillin in association with clavulanic acid, and derivatives or mixtures thereof.

27. The kit according to claim 26, wherein said first therapeutically active substance is carveol and said second therapeutically active substance is amoxicillin.

28. The kit according to claim 25, wherein said first therapeutically active substance is alpha-ionone or beta-ionone and said second therapeutically active substance is cephazolin.

29. The kit according to claim 25, wherein said first therapeutically active substance is thymol and said second therapeutically active substance is isoniazide.

30. A method for treating an infection caused by bacteria comprising the simultaneous or sequential administration to a patient of:
at least one first therapeutically active substance selected from the group consisting of carveol, thymol, eugenol, borneol, carvacrol, alpha-ionone, beta-ionone, and isomers, derivatives or mixtures thereof, and
at least one second therapeutically active substance which is an antibiotic selected from the group consisting of the beta-lactams, cephalosporins, tetracyclines, rifamycins, peptides, amino sides, phenicols, macrolides and derivatives or mixtures thereof.

31. The method according to claim 30, wherein said first therapeutically active substance is selected from the group consisting of carvacrol, carveol, thymol, alpha-ionone and beta-ionone, and said second therapeutically active substance is selected from the group consisting of amoxicillin, amoxicillin in association with clavulanic acid, ampicillin, streptomycin, erythromycin, polymyxin B, chloramphenicol, tetracycline, chlortetracycline, rifampicin, isoniazide, cephazolin, and derivatives or mixtures thereof.

32. The method according to claim 31, wherein said first therapeutically active substance is carveol or carvacrol and said second therapeutically active substance is selected from the group consisting of amoxicillin, ampicillin, chloramphenicol, tetracycline, chlortetracycline, streptomycin, erythromycin, polymyxin B, rifampicin, amoxicillin in association with clavulanic acid, and derivatives or mixtures thereof.

33. The method according to claim 32, wherein said first therapeutically active substance is carveol and said second therapeutically active substance is amoxicillin.

34. The method according to claim 31, wherein said first therapeutically active substance is alpha-ionone or beta-ionone and said second therapeutically active substance is cephazolin.

35. The method according to claim 31, wherein said first therapeutically active substance is thymol and said second therapeutically active substance is isoniazide.

36. The method according to claim 30, wherein said method comprises simultaneously or sequentially administering to a patient:
between 10 and 200 mg/kg of body weight/day of said first therapeutically active substance; and
between 2 and 100 mg/kg of body weight/day of said second therapeutically active substance.
